(19) United States
(12) Patent Application Publication
(10) Pub. No.: US 2022/0215952 A1
(43) Pub. Date: Jul. 7, 2022

(54) WIRELESS PATIENT HEALTH MONITORING AND MANAGEMENT SYSTEM USING IOT AND 6G TECHNOLOGY

(71) Applicants: Sami Bourouis, Taif (SA); Poongodi Manoharan, Doha (QA); Mithun Bhowmick, Durgapur (IN); Saiful Islam, Abha (SA); Abhishek Madduri, Durham, NC (US); Anjaneya Krishna Turai, Pimpri-Chinchwad (IN); Nithesh Derin Joan O, Chennai (IN)

(72) Inventors: Sami Bourouis, Taif (SA); Poongodi Manoharan, Doha (QA); Mithun Bhowmick, Durgapur (IN); Saiful Islam, Abha (SA); Abhishek Madduri, Durham, NC (US); Anjaneya Krishna Turai, Pimpri-Chinchwad (IN); Nithesh Derin Joan O, Chennai (IN)

(21) Appl. No.: 17/684,705

(22) Filed: Mar. 2, 2022

(51) Int. Cl. G16H 40/67; G16H 10/60; G16H 50/20; H04L 67/12

(52) U.S. Cl. CPC G16H 40/67; G16H 10/60; H04L 5/14; H04L 67/12; G16H 50/20

(57) ABSTRACT

The system comprises a plurality of sensing nodes for detecting real time health parameters of patients; a user interface having a plurality of entry fields allotted to a plurality of medical practitioners and pharmacy shops for updating an information of treatment, medication, disease, stage of disease, treatment duration, and the like; a 6G transceiver for wirelessly communicating with the plurality of sensing nodes and user interfaces; a blockchain-based memory for storing the real time health parameters and the information of treatment, medication, disease, stage of disease, treatment duration, and the like; and a central processing unit for generating a health record of each patients thereby storing into the blockchain-based memory such that a medical practitioner is allowed to access the stored data to study the health record of a patient before treatment or before suggesting any medication/vaccine to avoid the complications or any side effect.

### FIELD OF THE INVENTION

The present disclosure relates to smart healthcare systems, more particularly, a wireless patient health monitoring and management system using IoT and 6G Technology.

### BACKGROUND OF THE INVENTION

In various cases, it has been observed that a patient may get complications during a treatment due to their prior treatments or current medicines. In few cases, the medical practitioners do not think about or forget to ask about prior and ongoing treatments of the patients and suggest a medication, which results in complications. Sometime, the patient also forgot to inform about his/her allergy, which results in side effect and other complications.

Traditionally, the medical practitioners and pharmacy shops either does not make any health record of a patient or record in a paper which is destroyed or misplaced with respect to time. However, when the patient visit for next treatment, he/she have to explain their health issues again which may result in gap in information exchange.

Currently, there is no system available in the market for providing patient health record management. In the view of the forgoing discussion, it is clearly portrayed that there is a need to have a wireless patient health monitoring and management system using IoT and 6G Technology.

### SUMMARY OF THE INVENTION

The present disclosure seeks to provide a smart wireless patient health monitoring and management system for smart cities using internet of things and 6G-based blockchain technology.

In an embodiment, a wireless patient health monitoring and management system using IoT and 6G Technology is disclosed. The system includes a plurality of sensing nodes for detecting real time health parameters of patients within a range of area. The system further includes a user interface having a plurality of entry fields allotted to a plurality of medical practitioners and pharmacy shops for updating an information of treatment, medication, disease, stage of disease, treatment duration, and the like. The system further includes a sixth-generation (6G) transceiver for wirelessly communicating with the plurality of sensing nodes and user interfaces using a time division duplexing carrier. The system further includes a blockchain-based memory coupled to the 6G transceiver for storing the real time health parameters and the information of treatment, medication, disease, stage of disease, treatment duration, and the like. The system further includes a central processing unit for generating a health record of each patients thereby storing into the blockchain-based memory such that a medical practitioner is allowed to access the stored data to study the health record of a patient before treatment or before suggesting any medication/vaccine to avoid the complications or any side effect.

In another embodiment, the real time health parameters are selected from a group of body temperature, oxygen level, blood pressure, heart rate.

In another embodiment, the plurality of sensing nodes comprises: a monitoring band worn on a user body for monitoring heath parameters of the user; and a plurality of sensors installed on a commercial area including hospital, office, colleges and the like for detecting heath parameters of the user.

In another embodiment, the plurality of sensors are selected from a group of a temperature sensor, oxygen level sensor, blood pressure sensor, heart rate sensor, and the like, wherein the health parameters detected by the plurality of sensors are used when the person within the range of area is not having a monitoring band, wherein the plurality of sensors equipped with the monitoring band to solely detect the health parameters.

In another embodiment, the real time health parameters and the information of treatment, medication, disease, stage of disease, treatment duration, and the like of transmission is pre-processed through an artificial intelligence technique for removing noise and predicting vacant information of the transmission.

In another embodiment, the user interface is accessed by a registered medical practitioners and pharmacy shops, wherein an information is updated to the user interface for each time the patient receives any treatment or medication by any of the plurality of medical practitioners or pharmacy shops.

In another embodiment, the medical practitioners and pharmacy shops is registered on the user interface by entering personal credentials along with a medical certificate, wherein the medical practitioners and pharmacy shops are allowed to entry a new record into the health record of the person using the user interface, but not allowed to make any change in prior health record.

In another embodiment, an account of each of the patients are registered during one of a first treatment or first medication, wherein the patient is allowed to see his/her health records, but not allowed to make any changes in their health records.

In another embodiment, the information of treatment, medication, disease, stage of disease, treatment duration, and the like is filled into the plurality of entry fields of the user interface.

In another embodiment, the central processing unit comprises a convolution neural network technique-based model for generating a health record of each patients and for sending an alert to a particular medical practitioner in case of seriousness predicted by interpreting real time health parameters of a patient.

An object of the present disclosure is to develop a fast and secured healthcare data management system.

Another object of the present disclosure is to provide a health record of a patient to be studied before treatment or before suggesting any medication/vaccine to avoid the complications or any side effect.

Yet another object of the present invention is to deliver an expeditious and cost-effective a wireless patient health monitoring and management system using IoT and 6G Technology.

To further clarify advantages and features of the present disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a block diagram of a wireless patient health monitoring and management system using IoT and 6G Technology in accordance with an embodiment of the present disclosure;

FIG. 2 illustrates an architecture of the wireless patient health monitoring and management system using IoT and 6G Technology in accordance with an embodiment of the present disclosure; and

FIG. 3 illustrates a flow chart of a wireless patient health monitoring and management method using IoT and 6G Technology in accordance with an embodiment of the present disclosure.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

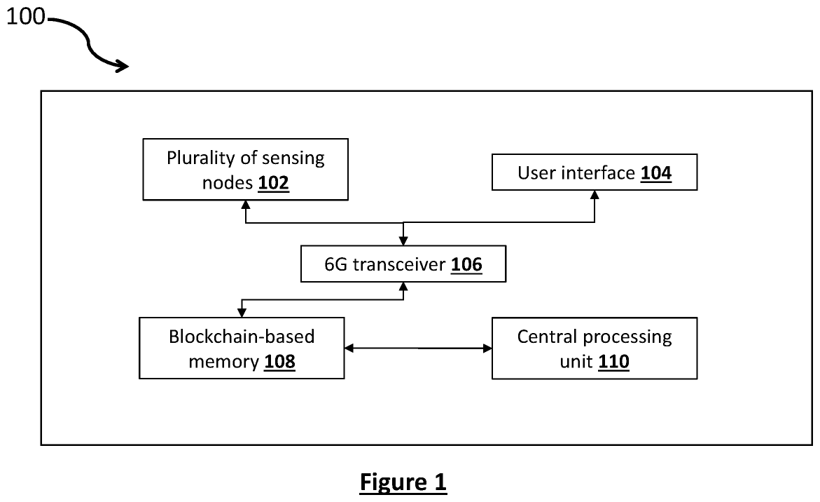

Referring to FIG. 1, a block diagram of a wireless patient health monitoring and management system using IoT and 6G Technology is illustrated in accordance with an embodiment of the present disclosure. The system 100 includes a plurality of sensing nodes 102 for detecting real time health parameters of patients within a range of area. The plurality of sensing nodes 102 are installed within a range of the area or coupled with the patient body.

In an embodiment, a user interface 104 is having a plurality of entry fields allotted to a plurality of medical practitioners and pharmacy shops for updating an information of treatment, medication, disease, stage of disease, treatment duration, and the like. The user interface 104 is installed into a user computing device. The user interface 104 is authenticated by entering user name and password allotted to the patient, registered medical practitioners and pharmacy shops after successful registration.

In an embodiment, a sixth-generation (6G) transceiver 106 is wirelessly connected to the plurality of sensing nodes 102 and the user interface 104 for wirelessly communicating with the plurality of sensing nodes 102 and user interfaces 104 using a time division duplexing carrier.

In an embodiment, a blockchain-based memory 108 is coupled to the 6G transceiver 106 for storing the real time health parameters and the information of treatment, medication, disease, stage of disease, treatment duration, and the like.

In an embodiment, a central processing unit 110 is connected to the blockchain-based memory 108 for generating a health record of each patients thereby storing into the blockchain-based memory 108 such that a medical practitioner is allowed to access the stored data to study the health record of a patient before treatment or before suggesting any medication/vaccine to avoid the complications or any side effect.

In another embodiment, the real time health parameters are selected from a group of body temperature, oxygen level, blood pressure, heart rate.

In another embodiment, the real time health parameters and the information of treatment, medication, disease, stage of disease, treatment duration, and the like of transmission is pre-processed through an artificial intelligence technique for removing noise and predicting vacant information of the transmission.

In another embodiment, the user interface 104 is accessed by a registered medical practitioners and pharmacy shops, wherein an information is updated to the user interface 104 for each time the patient receives any treatment or medication by any of the plurality of medical practitioners or pharmacy shops.

In another embodiment, the medical practitioners and pharmacy shops is registered on the user interface 104 by entering personal credentials along with a medical certificate, wherein the medical practitioners and pharmacy shops are allowed to entry a new record into the health record of the person using the user interface 104, but not allowed to make any change in prior health record.

In another embodiment, an account of each of the patients are registered during one of a first treatment or first medication, wherein the patient is allowed to see his/her health records, but not allowed to make any changes in their health records.

In another embodiment, the information of treatment, medication, disease, stage of disease, treatment duration, and the like is filled into the plurality of entry fields of the user interface 104.

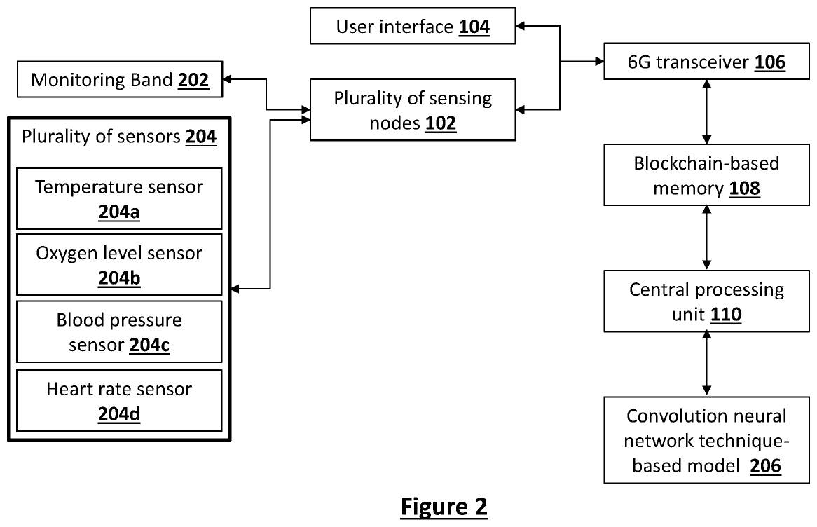

FIG. 2 illustrates an architecture of the wireless patient health monitoring and management system using IoT and 6G Technology in accordance with an embodiment of the present disclosure. The plurality of sensing nodes 102 includes a monitoring band 202 worn on a user body for monitoring heath parameters of the user. The plurality of monitoring bands 202 are allotted to the patients/people within the area for detecting heath parameters of the user

In an embodiment, a plurality of sensors 204 are installed on a commercial area including hospital, office, colleges and the like for detecting heath parameters of the user.

In another embodiment, the plurality of sensors 204 are selected from a group of a temperature sensor 204a, oxygen level sensor 204b, blood pressure sensor 204c, heart rate sensor 204d, and the like, wherein the health parameters detected by the plurality of sensors 204 are used when the person within the range of area is not having a monitoring band 202, wherein the plurality of sensors 204 equipped with the monitoring band 202 to solely detect the health parameters. However, the health parameters detected by the monitoring band 202 and plurality of sensors 204 can be compared to minimize error. In case, the data health parameters detected by the monitoring band 202 and plurality of sensors 204 does not match, the detection process is performed up to a predetermined iteration.

In one embodiment, the iterated health parameters are stored into the blockchain-based memory 108 through the 6G transceiver 106. The blockchain-based memory 108 is protected from editing and deleting of data.

In one embodiment, the iterated data is processed by the central processing unit 110 to generate the health record of each patients so that a medical practitioner is allowed to access the stored data to study the health record of a patient before treatment or before suggesting any medication/vaccine to avoid the complications or any side effect.

In another embodiment, the central processing unit 110 comprises a convolution neural network technique-based model 206 for generating a health record of each patients and for sending an alert to a particular medical practitioner in case of seriousness predicted by interpreting real time health parameters of a patient.

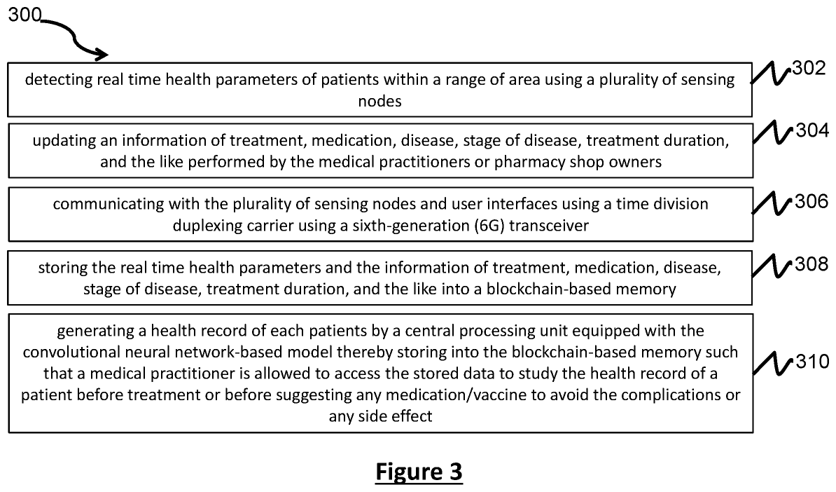

FIG. 3 illustrates a flow chart of a wireless patient health monitoring and management method using IoT and 6G Technology in accordance with an embodiment of the present disclosure. At step 302, the method 300 includes detecting real time health parameters of patients within a range of area using a plurality of sensing nodes 102.

At step 304, the method 300 includes updating an information of treatment, medication, disease, stage of disease, treatment duration, and the like performed by the medical practitioners or pharmacy shop owners using a user interface 104 having a plurality of entry fields allotted to a plurality of medical practitioners and pharmacy shops.

At step 306, the method 300 includes communicating with the plurality of sensing nodes 102 and user interfaces 104 using a time division duplexing carrier using a sixth-generation (6G) transceiver 106.

At step 308, the method 300 includes storing the real time health parameters and the information of treatment, medication, disease, stage of disease, treatment duration, and the like into a blockchain-based memory 108.

At step 310, the method 300 includes generating a health record of each patients by a central processing unit 110 equipped with the convolutional neural network-based model 206 thereby storing into the blockchain-based memory 108 such that a medical practitioner is allowed to access the stored data to study the health record of a patient before treatment or before suggesting any medication/vaccine to avoid the complications or any side effect.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A wireless patient health monitoring and management system using IoT and 6G Technology, the system comprises:
a plurality of sensing nodes for detecting real time health parameters of patients within a range of area;
a user interface having a plurality of entry fields allotted to a plurality of medical practitioners and pharmacy shops for updating an information of treatment, medication, disease, stage of disease, treatment duration, and the like;
a sixth-generation (6G) transceiver for wirelessly communicating with the plurality of sensing nodes and user interfaces using a time division duplexing carrier;
a blockchain-based memory coupled to the 6G transceiver for storing the real time health parameters and the information of treatment, medication, disease, stage of disease, treatment duration, and the like; and
a central processing unit for generating a health record of each patients thereby storing into the blockchain-based memory such that a medical practitioner is allowed to access the stored data to study the health record of a patient before treatment or before suggesting any medication/vaccine to avoid the complications or any side effect.

2. The system of claim 1, wherein the real time health parameters are selected from a group of body temperature, oxygen level, blood pressure, heart rate.

3. The system of claim 1, wherein the plurality of sensing nodes comprises:
a monitoring band worn on a user body for monitoring heath parameters of the user; and
a plurality of sensors installed on a commercial area including hospital, office, colleges and the like for detecting heath parameters of the user.

4. The system of claim 1, wherein the plurality of sensors are selected from a group of a temperature sensor, oxygen level sensor, blood pressure sensor, heart rate sensor, and the like, wherein the health parameters detected by the plurality of sensors are used when the person within the range of area is not having a monitoring band, wherein the plurality of sensors equipped with the monitoring band to solely detect the health parameters.

5. The system of claim 1, wherein the real time health parameters and the information of treatment, medication, disease, stage of disease, treatment duration, and the like of transmission is pre-processed through an artificial intelligence technique for removing noise and predicting vacant information of the transmission.

6. The system of claim 1, wherein the user interface is accessed by a registered medical practitioners and pharmacy shops, wherein an information is updated to the user interface for each time the patient receives any treatment or medication by any of the plurality of medical practitioners or pharmacy shops.

7. The system of claim 6, wherein the medical practitioners and pharmacy shops is registered on the user interface by entering personal credentials along with a medical certificate, wherein the medical practitioners and pharmacy shops are allowed to entry a new record into the health record of the person using the user interface, but not allowed to make any change in prior health record.

8. The system of claim 1, wherein an account of each of the patients are registered during one of a first treatment or first medication, wherein the patient is allowed to see his/her health records, but not allowed to make any changes in their health records.

9. The system of claim 1, wherein the information of treatment, medication, disease, stage of disease, treatment duration, and the like is filled into the plurality of entry fields of the user interface.

10. The system of claim 1, wherein the central processing unit comprises a convolution neural network technique-based model for generating a health record of each patients and for sending an alert to a particular medical practitioner in case of seriousness predicted by interpreting real time health parameters of a patient.
